SEQUENCE LISTING (53 sequences)

SEQ ID NO: 1 - DNA, 38 bp, Artificial Sequence
  Primer for PCR
caaactgcaggtgatgtcaaagcttgaaaaaacgcacg

SEQ ID NO: 2 - DNA, 39 bp, Artificial Sequence
  Primer for PCR
catatctagacgtgggcaatcatttgcatggatgacagc

SEQ ID NO: 3 - DNA, 40 bp, Artificial Sequence
  Primer for PCR
gctcggacagcgttctctatttccaataccgcaaatcacg

SEQ ID NO: 4 - DNA, 50 bp, Artificial Sequence
  Primer for PCR
cgcggaattcctaatgtgtgtttacgacaattctcacttcatacttttcc

SEQ ID NO: 5 - DNA, 265 bp, Staphylococcus aureus
gatcttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgcca

SEQ ID NO: 6 - DNA, 42 bp, Artificial Sequence
  Primer for PCR
tcctggatccgatcttctcaaaaaatactacctgtcccttgc

SEQ ID NO: 7 - DNA, 34 bp, Artificial Sequence
  Primer for PCR
cccgactagttggcacaaatgtgaggcattttcg

SEQ ID NO: 8 - DNA, 65 bp, Artificial Sequence
  Linker
gatcctatccgcggtcgactagtcataggcctgcagcccgcctaatgagcgggcttttttggtac

SEQ ID NO: 9 - DNA, 57 bp, Artificial Sequence
  Linker
caaaaaagcccgctcattaggcgggctgcaggcctatgactagtcgaccgcggatag

SEQ ID NO: 10 - DNA, 55 bp, Artificial Sequence
  Primer for PCR
ccgcggtaccgtataataaagaataattattaatctgtagacaaattgtgaaagg

SEQ ID NO: 11 - DNA, 46 bp, Artificial Sequence
  Primer for PCR
cttttgtttataagtgggtaaaccgtgaatatcgtgttcttttcac

SEQ ID NO: 12 - DNA, 64 bp, Artificial Sequence
  Primer for PCR
ggtattggaaatagagaacgctgtccgagccttttgtttataagtgggtaaaccgtgaatatcg

SEQ ID NO: 13 - DNA, 72 bp, Artificial Sequence
  Primer for PCR
taataactagtaaggaggacatatgaataaattagtaaaattaacagatttaaagcgcattttcaaagatgg

SEQ ID NO: 14 - DNA, 56 bp, Artificial Sequence
  Primer for PCR
gccgcacgcgttcatatatccataatctttaagttatctggaataattaaatctgc

SEQ ID NO: 15 - DNA, 60 bp, Artificial Sequence
  Primer for PCR
taataacgcgtaaggaggacatatgttagaaagtgaagtatctaaacaaattacaactcc

SEQ ID NO: 16 - DNA, 47 bp, Artificial Sequence
  Primer for PCR
gccgcaggcctttattttactgaaagataatcatgtacaaccttagg

SEQ ID NO: 17 - DNA, 61 bp, Artificial Sequence
  Primer for PCR
ggagtcactagtaaggaggacatatgaaaggttttgcaatgctaggtattaataagttagg

SEQ ID NO: 18 - DNA, 48 bp, Artificial Sequence
  Primer for PCR
atctcgagttataatataactactgctttaattaagtcttttggcttg

SEQ ID NO: 19 - DNA, 1296 bp, Artificial Sequence
  Fragment conferring chloramphenicol resistance from pHT01, MoBiTec GmbH, Germany.
cgatcggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaaaacgatacattaataggtacgaaaaagcaactttttttgcgcttaaaaccagtcataccaataacttaagcatgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgac

SEQ ID NO: 20 - DNA, 35 bp, Artificial Sequence
  Primer for PCR
tatattctcgagcgatcggcaatagttacccttat

SEQ ID NO: 21 - DNA, 33 bp, Artificial Sequence
  Primer for PCR
cagttatctagagtcgatttttgtgatgctcgt

SEQ ID NO: 22 - DNA, 37 bp, Artificial Sequence
  Primer for PCR
actggcatcaattgcgttattcgctggatttttattg

SEQ ID NO: 23 - DNA, 35 bp, Artificial Sequence
  Primer for PCR
atgaaggtaccgcgtactgcctgaacgagaagcta

SEQ ID NO: 24 - DNA, 30 bp, Artificial Sequence
  Primer for PCR
tagagtctagagcggaaatggtgtgaggtt

SEQ ID NO: 25 - DNA, 32 bp, Artificial Sequence
  Primer for PCR
agtaagcttaccatttagcacgtaatcaaagc

SEQ ID NO: 26 - DNA, 42 bp, Artificial Sequence
  Primer for PCR
tcctggtaccgatcttctcaaaaaatactacctgtcccttgc

SEQ ID NO: 27 - DNA, 60 bp, Artificial Sequence
  Primer for PCR
taatagctagcaaggaggacatatgagagatgtagtaatagtaagtgctgtaagaactgc

SEQ ID NO: 28 - DNA, 55 bp, Artificial Sequence
  Primer for PCR
gccgcaggccttaataacttagttatatataactatttagtctctttcaactacg

SEQ ID NO: 29 - DNA, 30 bp, Artificial Sequence
  Primer for PCR
tccgggatccaaaaaagcccgctcattagg

SEQ ID NO: 30 - DNA, 1179 bp, Artificial Sequence
  Thiolase from C. acetobutylicum in for B. subtilis codon optimized form
atgagggacgtagtaatagtaagcgcagtcagaacagccatcggagcttacggtaagacgctgaaagacgtcccggcaaccgagctgggcgctattgtcattaaagaagcagtgcgtcgtgcgaatatcaatccgaacgaaatcaatgaggtcatctttggtaatgttctgcaggcgggtctgggccagaacccagctcgtcaggctgccgttaaagcgggtctgccgctggaaaccccagcgttcacgatcaacaaagtgtgcggcagcggcttgcgtagcatttccctggcagcgcaaatcattaaggcaggtgacgccgacaccatcgtggtcggtggcatggagaatatgagccgcagcccgtatctgatcaacaaccaacgttggggccagcgtatgggtgacagcgagctggtggatgaaatgattaaggacggtctgtgggacgcattcaacggctatcacatgggtgttactgcagagaatattgcagagcagtggaacatcactcgcgaagagcaggatgagtttagcctgatgagccagcagaaggcggagaaggcgatcaagaatggtgagttcaaggacgagattgtgccagtcctgattaagacgaaaaagggcgagattgtctttgatcaagatgagttcccgcgttttggcaacaccattgaagcactgcgcaaattgaaaccgatcttcaaagagaatggtacggttaccgctggtaacgcaagcggcttgaatgatggtgcagccgcgctggtcattatgagcgcggacaaagccaacgcgctgggtatcaaaccgttggcgaagatcaccagctacggtagctacggtgtggacccgtccatcatgggttatggcgcgttctacgctaccaaagcggcgctggataagattaacttgaaaccggaggatctggatttgatcgaagcgaacgaagcctatgcaagccagagcattgcggtgacccgcgatctgaatctggacatgagcaaagttaacgtaaatggcggtgccattgcgctgggccacccgattggcgcaagcggtgctcgcatcctggttacgctgctgtacgcgatgcagaagcgtgactcgaagaagggtctggcaaccctgtgcatcggtggtggccagggcaccgcattggtcgttgagcgtgactaa

SEQ ID NO: 31 - DNA, 654 bp, Artificial Sequence
  CoA transferase subunit A from C. beijerinckii in for B. subtilis codon optimized form
atgaacaaattagtcaaactcacagacctaaaaagaatattcaaggatggcatgaccatcatggttggtggttttctggattgtggcacgccggagaacattattgatatgttggttgatctgaatatcaagaacctgactatcatctctaacgataccgcttttccgaacaaaggtatcggtaaactgattgttaatggccaagtcagcaaggttattgccagccacattggtacgaatcctgaaaccggtaagaagatgagcagcggtgagctgaaggttgaactgtctccgcagggcaccctgattgaacgcatccgtgctgcgggcagcggcctgggtggcgtcctgaccccgaccggtctgggtacgattgtggaagagggtaagaaaaaggtcaccattgatggtaaagaatacctgctggagctgccgctgagcgcagatgtgagcctgatcaaaggttctatcgtggacgaatttggtaacaccttctaccgtgcagcgaccaagaatttcaatccatacatggcgatggcggccaaaaccgtcattgtcgaagcggagaacctggttaagtgcgaagatctgaaacgcgatgccattatgactccgggcgtgctggttgattacatcgtgaaagaggcggcatga

SEQ ID NO: 32 - DNA, 666 bp, Artificial Sequence
  CoA transferase subunit B from C. beijerinckii in for B. subtilis codon optimized form
atgatcgtagacaaagtactggccaaggagataatagcaaaaagagtcgctaaggagctgaagaaagatcaactggtcaatctgggcattggcctgccaaccctggttgcgaactacgttccgaaggagatgaacatcaccttcgaaagcgagaacggtatggttggcatggcgcaaatggcgagctcgggtgagaatgacccggacatcattaatgcaggcggtgaatatgtcaccttgttgccacagggcagcttctttgactccagcatgagctttgcactgatccgtggcggccacgtcgatgttgctgtgctgggtgcgctggaagttgatgaaaaaggtaacttggcaaattggatcgtcccaaataagatcgttccgggtatgggtggtgccatggacctggcgattggtgcaaagaagatcattgttgcgatgcaacacacgggtaaaagcaaaccgaagattgtcaagaaatgtaccctgccgctgaccgcgaaagcgcaggttgacttgatcgttaccgaattgtgcgttatcgacgttaccaacgacggtctgctgctgaaggaaattcacaaagacactaccatcgacgagattaagttcttgaccgacgcggacctgatcatcccggacaacctgaagattatggatatctga

SEQ ID NO: 33 - DNA, 741 bp, Artificial Sequence
  Acetoacetate decarboxylase from C. beijerinckii in for B. subtilis codon optimized form
atgctcgaatcagaagtcagtaaacaaatcacaacgccgctcgcagcacctgcatttccgcgtggtccgtatcgttttcacaaccgtgaatacctgaacatcatttatcgtaccgatctggacgcgctgcgcaaaattgtgccggagccgttggaactggatcgtgcctacgtccgctttgagatgatggccatgccggataccactggtttgggttcctacacggagtgcggtcaagcgattccggttaagtacaatggtgtcaaaggtgattacctgcacatgatgtacctggataacgagccggcgattgcggtcggtcgcgagagcagcgcgtacccaaagaagctgggttatccgaagctgttcgttgactctgataccctggtcggtacgctgaagtatggcactctgccggtggcgaccgcgaccatgggttacaaacatgagccgctggatctgaaggaagcatacgcgcaaatcgcgcgtcctaacttcatgctgaaaatcatccagggctacgatggcaaaccgcgcatctgcgagctgatctgtgcagagaacaccgatatcacgatccacggcgcctggaccggtagcgcccgtctgcagctgttcagccacgcactggcaccgctggcagacctgccggtcctggagattgtgagcgcctcccacattctgactgacctgactctgggcaccccgaaagttgtccatgactatctgagcgttaagtaa

SEQ ID NO: 34 - DNA, 64 bp, Artificial Sequence
  Primer for PCR
catttgtgccaactagttaaggaggaaaaaaaatgagggacgtagtaatagtaagcgcagtcag

SEQ ID NO: 35 - DNA, 33 bp, Artificial Sequence
  Primer for PCR
cttaaagcttttagtcacgctcaacgaccaatg

SEQ ID NO: 36 - DNA, 76 bp, Artificial Sequence
  Primer for PCR
cgtgactaaaagctttaaggaggaaaaaaaatgaacaaattagtcaaactcacagacctaaaaagaatattcaagg

SEQ ID NO: 37 - DNA, 26 bp, Artificial Sequence
  Primer for PCR
cttaacgcgttcatgccgcctctttc

SEQ ID NO: 38 - DNA, 60 bp, Artificial Sequence
  Primer for PCR
gcggcatgaacgcgttaaggaggaaaaaaaatgatcgtagacaaagtactggccaaggag

SEQ ID NO: 39 - DNA, 38 bp, Artificial Sequence
  Primer for PCR
cttagctagctcagatatccataatcttcaggttgtcc

SEQ ID NO: 40 - DNA, 65 bp, Artificial Sequence
  Primer for PCR
gatatctgagctagctaaggaggaaaaaaaatgctcgaatcagaagtcagtaaacaaatcacaac

SEQ ID NO: 41 - DNA, 46 bp, Artificial Sequence
  Primer for PCR
tgcatgcctgcaggtcgacttacttaacgctcagatagtcatggac

SEQ ID NO: 42 - DNA, 1056 bp, Artificial Sequence
  Isopropyl alcohol dehydrogenase from C. beijerinckii in for B. subtilis codon optimized form
atgaagggcttcgcaatgctagggataaacaaattaggatggatcgaaaaggagcgtccagtagctggttcgtatgacgcaatcgtgcgtccgctggcggtcagcccgtgtaccagcgacattcacaccgttttcgagggtgcgttgggtgatcgcaagaatatgattctgggtcatgaagcggtgggcgaggtggttgaggtgggtagcgaagtgaaggatttcaagccgggtgatcgtgtgatcgtcccttgcaccacgccggattggcgtagcctggaggtgcaagcaggttttcaacaacatagcaacggcatgctggcgggttggaagttttccaacttcaaagatggcgttttcggtgagtatttccatgttaacgatgccgatatgaatctggcgattctgccgaaggacatgccgctggagaacgcggtcatgattaccgatatgatgaccaccggctttcatggcgcagagctggcggatatccagatgggtagcagcgtggtagttattggtattggcgccgttggcctgatgggtattgcgggtgcgaagctgcgtggtgccggtcgcatcattggtgttggtagccgcccaatctgtgttgaagcggccaagttctacggtgcgacggatattctgaattacaagaacggccatattgtggaccaagtgatgaaactgactaacggcaaaggtgttgaccgtgtcattatggcaggtggcggcagcgaaacgctgagccaggccgtgtccatggttaaaccgggtggtatcatttccaacatcaattaccacggtagcggtgatgcactgctgattccgcgtgtggaatggggctgtggtatggcccataagaccattaaaggtggtctgtgcccgggtggtcgtctgcgtgcggagatgctgcgtgatatggtggtgtataaccgtgtggacctgagcaagctggttacccacgtgtatcacggctttgaccacatcgaggaagcactgttgctgatgaaagacaaaccaaaagatctgattaaagcggtggtgattctgtaa

SEQ ID NO: 43 - DNA, 114 bp, Artificial Sequence
  Untranslated region from C. beijerinckii
cataatagatcatggaggattatacatgtacaagatagttagtaaaaaggagctaacaaacaatatattctcaatggatatagaagctccaagagtagcaaaatctgcaggcct

SEQ ID NO: 44 - DNA, 39 bp, Artificial Sequence
  Primer for PCR
taaatataaatcggatccacaatcggcaattgacgaaac

SEQ ID NO: 45 - DNA, 46 bp, Artificial Sequence
  Primer for PCR
atactatgtcgacccaacatgattaacaattattagaggtcatcgt

SEQ ID NO: 46 - DNA, 34 bp, Artificial Sequence
  Primer for PCR
gctgaattcaaccaatcaccatttgatattctgt

SEQ ID NO: 47 - DNA, 34 bp, Artificial Sequence
  Primer for PCR
tatggatccgtcctttatcttgtaaagcgtcaaa

SEQ ID NO: 48 - DNA, 38 bp, Artificial Sequence
  Primer for PCR
atactatgtcgacgtttttgactatgtgcttgaggatt

SEQ ID NO: 49 - DNA, 37 bp, Artificial Sequence
  Primer for PCR
tatgttgcatgctattatcgcagtgaaaaccaataca

SEQ ID NO: 50 - DNA, 39 bp, Artificial Sequence
  Primer for PCR
tagagaagcttgcaggaaacaaagtgctgaatccgattg

SEQ ID NO: 51 - DNA, 37 bp, Artificial Sequence
  Primer for PCR
tagagggatccgcagcatcaaattggaattcgccgtc

SEQ ID NO: 52 - DNA, 47 bp, Artificial Sequence
  Primer for PCR
gtacaagcttatgagcaatattacgatctacgatgtagcgagagaag

SEQ ID NO: 53 - DNA, 44 bp, Artificial Sequence
  Primer for PCR
gtttggatccgcagtgcttcgagtccggaatcatatgtgtaatc

(19) United States
(12) Patent Application Publication
(10) Pub. No.: US 2014/0273122 A1
(43) Pub. Date: Sep. 18, 2014

(54) NOVEL MICROORGANISM OF THE GENUS BACILLUS

(71) Applicant: MITSUI CHEMICALS, INC., Tokyo (JP)

(72) Inventors: Dominik Lukas JÜRGEN-LOHMANN, Singapore (SG); Su Sun Chong, Singapore (SG); Anjali Madhavan, Singapore (SG); Katsuyuki Takahashi, Singapore (SG)

(73) Assignee: MITSUI CHEMICALS, INC., Tokyo (JP)

(21) Appl. No.: 13/832,039

(22) Filed: Mar. 15, 2013

(51) Int. Cl. C12P 7/28; C12P 7/04; C12N 15/75

(52) U.S. Cl. CPC C12P 7/28; C12N 15/75; C12P 7/04
USPC 435/150; 435/252.31; 435/157

(57) ABSTRACT

The invention provides a microorganism of the genus Bacillus, including one or more gene(s) selected from a group consisting of a gene conferring thiolase activity, a gene conferring CoA transferase activity and a gene conferring acetoacetate decarboxylase activity, wherein the microorganism produces acetone.

### TECHNICAL FIELD

This invention relates to a microorganism of the genus Bacillus and a method of producing acetone or isopropyl alcohol using the same.

### BACKGROUND ART

Several descriptions of acetone or isopropyl alcohol producing bacteria exist in the literature. There are two main strategies for microbial acetone or isopropyl alcohol production:

1) Production using microbes based on naturally occurring acetone or isopropyl alcohol producing microbes.

2) Production using genetically modified microbes that do not produce acetone or isopropyl alcohol naturally.

Examples for the first strategy are described in Patent Literature 1 and 2. However, in these two examples, isopropyl alcohol is produced by Clostridium only alongside other solvents such as ethanol and butanol. Selectivity of the naturally occurring biocatalyst is low and purification of isopropyl alcohol is expected to be challenging.

Therefore, generally, the second strategy is preferred as it is thought to have the potential for a higher selectivity of the biocatalyst.

The second strategy is described for producing acetone using E. coli in Non Patent Literature 1. Through expression of thiL, ctfAB and adc from Clostridium acetobutylicum in the E. coli host cell acetone accumulated in the culture broth.

The production of isopropyl alcohol using the second strategy in E. coli has been described for example in Patent Literature 3, Non Patent Literatures 2 and 3. All three examples disclose a recombinant E. coli which can produce isopropyl alcohol. The recombinant E. coli expresses heterologous or homologous genes corresponding to thiolase, CoA transferase, acetoacetate decarboxylase and isopropyl alcohol dehydrogenase activities.

Additionally, Patent Literature 3 and Non Patent Literature 3 disclose the use of aeration and volatile capture to improve isopropyl alcohol production and recovery.

To the knowledge of the inventors, none of the described technologies is efficient or cost effective enough to result in commercial production. Therefore, there is a need to develop alternative acetone or isopropyl alcohol production technology.

Bacilli are more closely related to the natural producers of acetone or ispopropyl alcohol such as C. beijerinckii. In contrast to E. coli, Bacilli are also able to natively utilize carbon sources that are more cost effective than glucose, such as glycerol or starch, in an efficient manner. Moreover, e.g. Bacillus subtilis was found to show good tolerance to isopropyl alcohol in a study carried out by Non Patent Literature 4. Furthermore, many Bacilli share a number of convenient characteristics with E. coli such as the amenability to genetic manipulation, the ability to grow at various degree of oxygen supply and ease of cultivation. Lastly, many Bacilli such as Bacillus subtilis and Bacillus megaterium are generally regarded as safe (GRAS) in contrast to E. coli or Clostridia.

The abovementioned characteristics of Bacilli appear to make them promising candidates for development of alternative acetone or isopropyl alcohol production hosts. However, the only description known to the inventors that is showing reconstruction of a Clostridial solvent pathway in Bacilli resulted in insufficient production of butanol at 23 mg L⁻¹ (Non Patent Literature 5).

### CITATION LIST

### Patent Literature

[PTL 1] Chinese Patent Application Publication No. CN 1043956 A
[PTL 2] Japanese Patent Application Publication No. S61-67493
[PTL 3] WO2009/008377

### Non Patent Literature

[NPL 1] L. Bermejo et al., Applied and Environmental Microbiology, 1998, 64(3), 1079-1085
[NPL 2] T. Hanai et al., Applied and Environmental Microbiology, 2007, 73(24), 7814-7818
[NPL 3] K. Inokuma et al., Journal of Bioscience and Bioengineering, 2010, 110(6), 696-701
[NPL 4] Kataoka et al., AMB Express, 2011, 1:10
[NPL 5] Nielsen et al., Metabolic Engineering, 2009, 11(4-5), 262-273

### SUMMARY OF INVENTION

### Technical Problem

As described above, examples for production of acetone or isopropyl alcohol using genetically modified bacteria exist for E. coli. However, production efficiency is not good enough for a commercial process. On the other hand, solvent production using Clostridial pathways in Bacillus did not result in efficient production of solvent in these promising production host. The problem that there exists no efficient acetone or isopropyl alcohol producing Bacillus is to be solved by this invention.

### Solution to Problem

The first aspect of this invention is a microorganism of the genus Bacillus, including one or more gene(s) selected from a group consisting of a gene conferring thiolase activity, a gene conferring CoA transferase activity and a gene conferring acetoacetate decarboxylase activity, wherein the microorganism produces acetone.

The second aspect of this invention is a method for producing acetone or isopropyl alcohol using the above mentioned microorganism of the genus Bacillus.

### Advantageous Effects of Invention

According to this invention, a microorganism of the genus Bacillus that is capable of efficiently producing acetone or isopropyl alcohol can be provided.

### DESCRIPTION OF EMBODIMENTS

The acetone or isopropyl alcohol producing Bacillus of this invention to which 1 to 4 of a thiolase activity and/or CoA transferase activity and/or acetoacetate decarboxylase activity and/or isopropyl alcohol dehydrogenase activity have been imparted to, is capable of producing acetone or isopropyl alcohol from a carbon source. In preferable embodiments, the 1 to 4 activities are taken from bacteria of the genus Clostridium and are either introduced into the genome of the Bacillus or provided on a plasmid with a 4 nucleotide ribosome binding site to start codon spacing.

The method of producing acetone or isopropyl alcohol of this invention includes using the Bacillus described above to produce these compounds from a carbon source. In preferable embodiments, the Bacillus bacterium is cultured in an appropriate medium while supplying a gas to the culturing mixture and collecting the acetone or isopropyl alcohol produced by the culturing mixture.

In the invention, the carbon source is not particularly restricted as long as it can be metabolized by the acetone or isopropyl alcohol producing Bacillus of the invention. The carbon source may refer to any carbon source that can be used by the Bacillus of the invention naturally, e.g. starch, sucrose, glucose, arabinose and other saccharides as well as glycerin or fatty acids.

Up to four kinds of acetone or isopropyl alcohol-producing activities, that is, an acetoacetate decarboxylase activity, an isopropyl alcohol dehydrogenase activity, a CoA transferase activity and a thiolase activity are imparted to the acetone or isopropyl alcohol-producing Bacillus of the invention.

Additionally, the natural activities may be enhanced by imparting non-natural additional activities to the Bacillus bacterium. In the invention, “impart(ing)” an activity, in addition to introducing a gene encoding an enzyme from the outside of a host bacterium into the inside thereof, also includes enhancing the activity of a promoter an enzyme gene retained in the genome of a host bacterium, and replacing a promoter with another promoter to cause overexpression of an enzyme gene.

In nature, 61 different codons code for 20 amino acids. Therefore, in most cases more than one codon can code for a given amino acid, i.e. the genetic code is degenerate. It is well known to any person skilled in the art that altering the coding sequence without altering the resulting polypeptide sequence can effect the expression level of a gene. This is because a given microorganism may have a preference towards certain codons for certain amino acids, which is termed codon bias. Adapting a given codon sequence to a given codon bias is termed codon optimization. In this invention, coding sequences may be identical or similar to the original coding sequence or they may be altered in any way through codon optimization as long as they encode for an identical, essentially identical or functionally identical polypeptide. Additionally, coding sequences may be altered for other reasons such as for example to control mRNA secondary structure, nucleotide sequence motives, GC content etc. as long as they encode for an identical, essentially identical or functionally identical polypeptide.

Acetoacetate decarboxylase as referred to in the invention is the collective name of enzymes which are classified with the enzyme code 4.1.1.4 according to the report by the International Union of Biochemistry (I.U.B.) Enzyme Commission and catalyze reactions producing acetone from acetoacetic acid. Examples of such enzymes include those derived from bacteria of the genus Clostridium such as Clostridium acetobutylicum and Clostridium beijerinckii; and those derived from bacteria of the genus Bacillus such as Bacillus polymyxa. As the gene of acetoacetate decarboxylase which is introduced into the Bacillus of the invention, a DNA having a base sequence of the gene encoding acetoacetate decarboxylase obtained from each of the above-mentioned organisms, or a synthetic DNA sequence synthesized based on a known base sequence(s) of the gene may be used.

Preferable examples of the gene include those derived from bacteria of the genus Clostridium or bacteria of the genus Bacillus, and examples thereof include DNAs having a base sequence of the gene derived from Clostridium acetobutylicum, Clostridium beijerinckii or Bacillus polymyxa. A synthetic DNA having the amino acid sequence of the gene derived from Clostridium beijerinckii that has been optimized for codon usage in Bacillus is especially preferable.

Isopropyl alcohol dehydrogenase as referred to in the invention is the collective name of enzymes which are classified with the enzyme code 1.1.1.80 according to the report by the International Union of Biochemistry (I.U.B.) Enzyme Commission and catalyze reactions producing isopropyl alcohol from acetone. Examples of such enzymes include those derived from bacteria of the genus Clostridium such as Clostridium beijerinckii.

As the gene of isopropyl alcohol dehydrogenase which is introduced into the host Bacillus employed in the invention, a DNA having a base sequence of the gene encoding isopropyl alcohol dehydrogenase obtained from each of the above-mentioned organisms or a synthetic DNA sequence synthesized based on a known base sequence(s) of the gene may be used. Preferable examples of the gene include those derived from bacteria of the genus Clostridium, and more preferable examples thereof include synthetic DNAs having an amino acid sequence of the gene derived from Clostridium beijerinckii that have been optimized for codon usage in Bacillus.

CoA transferase as referred to in the invention is the collective name of enzymes which are classified as the enzyme code 2.8.3.8 according to the report by the International Union of Biochemistry (I.U.B.) Enzyme Commission and catalyze reactions producing acetoacetic acid from acetoacetyl-CoA. Examples of such enzymes include those derived from bacteria of the genus Clostridium such as Clostridium acetobutylicum and Clostridium beijerinckii, bacteria of the genus Roseburia such as Roseburia intestinalis, bacteria of the genus Faecalibacterium such as Faecalibacterium prausnitzii, bacteria of the genus Coprococcus, trypanosomes such as Trypanosoma brucei and bacteria of the genus Escherichia such as Escherichia coli (E. coli). As the gene of CoA transferase which is introduced into the host Bacillus of the invention, a DNA having a base sequence of the gene encoding CoA transferase obtained from each of the above-mentioned organisms or a synthetic DNA sequence synthesized based on a known base sequence(s) of the gene may be used. Preferable examples of the gene include DNAs having a base sequence of the gene derived from bacteria of the genus Clostridium such as Clostridium acetobutylicum, bacteria of the genus Roseburia such as Roseburia intestinalis, bacteria of the genus Faecalibacterium such as Faecalibacterium prausnitzii, bacteria of the genus Coprococcus, trypanosomes such as Trypanosoma brucei and bacteria of the genus Escherichia such as Escherichia coli. More preferable examples thereof include those derived from bacteria of the genus Clostridium and bacteria of the genus Escherichia. A synthetic DNA having an amino acid sequence of the gene derived from Clostridium beijerinckii that has been optimized for codon usage in Bacillus is especially preferable.

Thiolase as referred to in the invention is the collective name of enzymes which are classified as the enzyme code 2.3.1.9 according to the report by the International Union of Biochemistry (I.U.B.) Enzyme Commission and catalyze reactions producing acetoacetyl-CoA from acetyl-CoA. Examples of such enzymes include those derived from bacteria of the genus Clostridium such as Clostridium acetobutylicum and Clostridium beijerinckii, bacteria of the genus Escherichia such as Escherichia coli, bacteria of the genus Bacillus such as B. subtilis and B. megaterium, bacteria of Halobacterium sp., bacteria of the genus Zoogloea such as Zoogloea ramigera, bacteria of Rhizobium sp., bacteria of the genus Bradyrhizobium such as Bradyrhizobium japonicum, bacteria of the genus Caulobacter such as Caulobacter crescentus, bacteria of the genus Streptomyces such as Streptomyces collinus, bacteria of the genus Enterococcus such as Enterococcus faecalis, yeasts of the genus Candida such as Candida tropicalis.

As the gene of thiolase which is introduced into the host Bacillus used in the invention, a DNA having a base sequence of the gene encoding thiolase obtained from each of the above-mentioned organisms or a synthetic DNA sequence synthesized based on a known base sequence(s) of the gene may be used. Preferable examples of the gene include DNAs having a base sequence of the gene derived from bacteria of the genus Clostridium such as Clostridium acetobutylicum and Clostridium beijerinckii, bacteria of the genus Bacillus such as B. subtilis and B. megaterium, bacteria of the genus Escherichia such as Escherichia coli, bacteria of Halobacterium sp., bacteria of the genus Zoogloea such as Zoogloea ramigera, bacteria of Rhizobium sp., bacteria of the genus Bradyrhizobium such as Bradyrhizobium japonicum, bacteria of the genus Caulobacter such as Caulobacter crescentus, bacteria of the genus Streptomyces such as Streptomyces collinus, bacteria of the genus Enterococcus such as Enterococcus faecalis, yeasts of the genus Candida such as Candida tropicalis. More preferable examples thereof include those derived from bacteria of the genus Clostridium and bacteria of the genus Bacillus; and a synthetic DNA having an amino acid sequence of the gene derived from Clostridium acetobutylicum that has been optimized for codon usage in Bacillus is especially preferable.

Among these, each of the above four kinds of enzymes is preferably derived from at least one species selected from the group consisting of bacteria of the genus Clostridium, bacteria of the genus Bacillus and bacteria of the genus Escherichia in view of the enzyme activity, and, in particular, more preferable are the cases where acetoacetate decarboxylase and isopropyl alcohol dehydrogenase are derived from a bacterium/bacteria of the genus Clostridium and the CoA transferase activity and thiolase activity are derived from a bacterium/bacteria of the genus Bacillus or Escherichia, or the cases where all of these four kinds of enzymes are derived from a bacterium/bacteria of the genus Clostridium.

The activity of each of these enzymes in the invention may be introduced from the outside of the host bacterium into the inside of the host bacterium, or alternatively, the activity of each of these enzymes in the invention may be realized by overexpression of the enzyme genes by enhancement of activity of a promoter(s) of the enzyme genes retained in the genome of the host bacterium or replacement of the promoter(s) with another promoter(s) to cause overexpression of the enzyme gene.

Introduction of the enzyme activities may be carried out, for example, by introduction of the genes encoding those four kinds of enzymes from the outside of the host bacterium into the inside of the host bacterium using gene recombination technology. In this case, the introduced enzyme genes may be either the same or different from the species of the host cell. Preparation of the genomic DNA, cleavage and ligation of a DNA, transformation, PCR (Polymerase Chain Reaction), design and synthesis of oligonucleotides used as primers, and the like may be carried out by the conventional methods well-known by persons skilled in the art. These methods are described in, for example, Sambrook, J., et al., “Molecular Cloning A Laboratory Manual, Second Edition”, Cold Spring Harbor Laboratory Press (1989).

Any promoter may be used as the promoter used for enhancement of the promoter activity or overexpression of the enzyme gene as long as it can be expressed in a host such as Bacillus subtilis or Bacillus megaterium.

For example, promoters derived from Bacillus subtilis or S. aureus or any other gram positive microorganism may be used. As examples the cggR promoter or amyE promoter from Bacillus subtilis may be used. More preferably inducible strong promoters such as the hyper spank promoter may be used and even more preferably, strong constitutive promoters such as the HPAII promoter from S. aureus plasmid pUB110 may be used.

These promoters may be introduced into the host cell according to a conventional method such that the target enzyme gene may be expressed by, for example, ligating the promoter(s) with a vector which is the same one with which the target enzyme gene is ligated, followed by introduction of the vector into the host cell together with the enzyme gene.

A ribosome binding site preceding the coding sequence of the gene to be introduced may be added. The ribosome binding site sequence can be any sequence that allows binding of the ribosome complex for subsequent translation in Bacillus. Preferably, the sequence should have some similarity to the ribosome binding site consensus sequence well known to a person skilled in the art. More preferably, the sequence should be AAGGAGG.

The introduction of genes into the host Bacillus may be carried out using a plasmid system or genomic integration. Plasmid vectors can be any vector that can be maintained in a Bacillus cell including pHY300 PLK, pUB110, pGDV1 and others.

Genomic integration can be carried out using integration vectors such as pMUTIN4 or by introducing a DNA sequence of interest preceded and followed by a DNA sequence homologous to the Bacillus genome. Sites for genomic integration may be chosen to be non-essential sites, such as lacA or amyE or sites that carry a specific function desired to be affected.

In certain embodiments where a specific function is to be affected, genomic integration may be carried out to positively affect acetone or isopropyl alcohol formation. This may relate to the attenuation or destruction of a specific function. Preferably this function relates to the formation of byproducts, such as acetolactate synthase, alpha acetolactate decarboxylase, pyruvate oxidase, phosphotransacetylase or carbon catabolite control protein. A.

Acetolactate synthase, (hereinafter may be referred to as alsS) in the invention is classified into the enzyme number [2.2.1.6], based on the report of the enzyme committee of International Union of Biochemistry (I.U.B.), and refers to an enzyme which reversibly catalyzes a reaction to produce acetoin from pyruvic acid.

Alpha acetolactate decarboxylase (hereinafter may be referred to as alsD) in the invention is classified into the enzyme number [4.1.1.5], based on the report of the enzyme committee of International Union of Biochemistry (I.U.B.), and refers to an enzyme which reversibly catalyzes a reaction to produce 2,3-butanediol from acetoin.

Pyruvate oxidase (hereinafter may be referred to as ydaP) in the invention is classified into the enzyme number [1.2.3.3], based on the report of the enzyme committee of International Union of Biochemistry (I.U.B.), and refers to an enzyme which reversibly catalyzes a reaction to produce acetate from pyruvic acid.

Phosphotransacetylase (hereinafter may be referred to as pta) in the invention is classified into the enzyme number [2.3.1.8], based on the report of the enzyme committee of International Union of Biochemistry (I.U.B.), and refers to an enzyme which reversibly catalyzes a reaction to produce acetate from acetyl-coA.

Carbon catabolite control protein A (hereinafter may be referred to as ccpA in the invention refers to a protein which the central regulator of carbon catabolite repression (CCR) in Bacillus megaterium, Bacillus subtilis, and other gram-positive bacteria of low G+C content.

In the invention, one or more enzyme activity selected from a group consisting of alsS activity, alsD activity, ydaP, pta and ccpA activity is inactivated as compared to that of the host Bacillus.

Inactivation of functions of an enzyme alsS, alsD, ydaP, pta or ccpA in the invention means complete loss of activity of the enzyme. In order to inactivate function of the enzyme, there are methods such as introducing a mutation into the gene encoding the protein, eliminating the gene, adding a drug which specifically inactivates the enzyme, irradiating the enzyme with ultraviolet rays or the like.

The alsSD operon consists of alsS and alsD enzyme units that code for acetolactate synthase and acetolactate decarboxylase respectively. Inactivation of the alsSD operon function can be carried out by inactivation of alsS, alsD or both.

The technique to carry out genome introduction or plasmid transformation is considered to be well known to any person skilled in the art.

Any combination of plasmid and/or genome introduction may be suitable in certain embodiments of the invention. For the acetone or isopropyl alcohol producing Bacillus it is preferable (a) to integrate related gene(s) into the chromosome of the host bacterium, or (b) to impart related genes in plasmid form with a spacer of four nucleotides between the abovementioned ribosome binding site sequence and the start codon of the related genes. More preferable, the genes are introduced into the genome in a non-essential locus. Even more preferable, the genes are introduced into the genome with a spacer of seven nucleotides between the abovementioned ribosome binding site sequence and the start codon of the related genes in the non-essential lacA locus.

In the invention, the host bacterium is a Bacillus which is used for introduction of the genes encoding the up to four kinds of enzymes of acetoacetate decarboxylase, isopropyl alcohol dehydrogenase, CoA transferase and thiolase, or a Bacillus which is the target of either enhancement of activity of promoter(s) of these enzymes or replacement of the promoter(s). Examples of such a Bacillus include Bacillus subtilis and Bacillus megaterium, which are especially convenient and have yielded plenty of results in industrial uses, and are preferably used.

The method for production of acetone or isopropyl alcohol of the invention includes producing acetone or isopropyl alcohol from a suitable carbon source using the acetone or isopropyl-alcohol producing Bacillus of the invention. This production method includes a method including assimilating the suitable carbon source by culturing the acetone or isopropyl alcohol-producing Bacillus in a mixture containing the acetone or isopropyl alcohol-producing Bacillus and the carbon source, and, after a certain period of time, purifying the acetone or isopropyl alcohol secreted in the culture medium using a known technique(s) such as distillation, membrane separation and extraction.

The mixture in the production method of acetone or isopropyl alcohol may contain mainly a basal medium generally used for culturing Bacilli, and any medium may be used as long as it is a medium normally used depending on the type of the acetone or isopropyl alcohol-producing Bacillus. Such a basal medium is not particularly limited as long as it is a medium containing a carbon source, nitrogen source, inorganic ions and, as required, other minor components. Preferably, the medium consists of a C medium base.

In addition, the culture medium may contain other additive components which are usually added to media for microorganisms, such as antibiotics, at concentrations at which they are usually used. An antifoaming agent is preferably added to the culture medium at an appropriate amount for suppressing foaming.

There is no particular limitation to the culture condition employed for the culturing in the invention. In embodiments, the culturing may be carried out under an aerobic condition with appropriately controlling pH and temperature so that the pH becomes in the range of 4 to 9, preferably in the range of 6 to 8, and the temperature becomes in the range of 20 to 50 degrees Celsius, preferably in the range of 25 to 42 degrees Celsius.

The method for collecting the acetone or isopropyl alcohol produced by the Bacillus of the invention may be carried out according to Patent Literature 3. More specifically, an aeration of 0.3 v/vm at agitation of 550 rpm is preferable. As a capturing setup, it is preferable to use two capturing vessels with 800 mL water as a capturing liquid each.

The present invention provides bacteria of the genus Bacillus capable of producing acetone and/or isopropyl alcohol and methods for producing the same using these bacteria.

Production of acetone and/or isopropyl alcohol is accomplished by introducing activities related to isopropyl alcohol production, namely a thiolase activity, a CoA transferase activity, an acetoacetate decarboxylase activity and an isopropyl alcohol dehydrogenase activity, into a Bacillus bacterium. Most preferably, the genes related to these activities are introduced into the chromosome of the Bacillus bacterium. If, however, the genes are to be provided on a plasmid, the spacing between ribosome binding site and initiation codon should be an unusual 4 nucleotides in length.

Additionally, production of acetone and/or isopropyl alcohol can be increased through inactivation of byproduct formation pathway genes alsS and/or alsD and/or pta. Formation of byproduct can also be prevented through inactivation of carbon catabolite repression protein ccpA.

Furthermore, it was surprisingly discovered in the process of the invention that Bacillus megaterium DSM319 is a natural producer of acetone.

Methods for producing acetone and/or isopropyl alcohol using the described Bacillus bacteria are also provided.

### Examples 1

### Construction of Vectors and Genome Integration Fragments

Integration of heterologous genes into the Bacillus subtilis 168 genome was carried out into loci that are considered to be non-essential under the given culture conditions. Namely, the lacA locus and amyE locus were used. For chromosomal integration, the fragment to be integrated was flanked by ca. 500 to 1000 bp regions termed F and R regions that are homologous to the Bacillus subtilis 168 genome and determine the site of integration through homologous recombination.

For introduction into lacA locus, a plasmid pBS2 was constructed. The plasmid is based on pUC18 and was constructed using standard molecular biology techniques. pUC18 can be obtained from e.g. Takara Bio Inc., Japan. The lacA F and R fragments of ca. 1000 bp each were amplified from Bacillus subtilis 168 genomic DNA using CAAACTGCAGGTGATGTCAAAGCTTGAAAAAACGCACG (SEQ ID NO: 1) and CATATCTAGACGTGGGCAATCATTTGCATGGATGACAGC (SEQ ID NO: 2) as well as GCTCGGACAGCGTTCTCTATTTCCAATACCGCAAATCACG (SEQ ID NO: 3) and CGCGGAATTCCTAATGTGTGTTTACGACAATTCTCACTTCATACTTTTCC (SEQ ID NO: 4), respectively. Bacillus subtilis 168 can be obtained from the Bacillus Genetic Stock Center, USA. Promoter element PHPAII of ca. 300 bp (SEQ ID NO: 5) was amplified from pUB110 using TCCTGGATCCGATCTTCTCAAAAAATACTACCTGTCCCTTGC (SEQ ID NO: 6) and CCCGACTAGTTGGCACAAATGTGAGGCATTTTCG (SEQ ID NO: 7). pUB110 can be obtained from the Bacillus Genetic Stock Center, USA. A linker containing a BamHI, SacII, SpeI site, a terminator sequence TRPA1 and a KpnI site was constructed using oligonucleotides GATCCTATCCGCGGTCGACTAGTCATAGGCCTGCAGCCCGCCTAATGAGCGGGCTTTTTTGGTAC (SEQ ID NO: 8) and CAAAAAAGCCCGCTCATTAGGCGGGCTGCAGGCCTATGACTAGTCGACCGCGGATAG (SEQ ID NO: 9). A sequence conferring resistance to spectinomycin of ca. 1200 bp was amplified from pIC156 using CCGCGGTACCGTATAATAAAGAATAATTATTAATCTGTAGACAAATTGTGAAAGG (SEQ ID NO: 10) and CTTTTGTTTATAAGTGGGTAAACCGTGAATATCGTGTTCTTTTCAC (SEQ ID NO: 11). pIC156 can be obtained from the Bacillus Genetic Stock Center, USA. The sequence of the spectinomycin fragment is described in GenBank accession number AB666466.1. This spectinomycin fragment was connected to lacA R by SOE PCR using oligo nucleotide GGTATTGGAAATAGAGAACGCTGTCCGAGCCTTTTGTTTATAAGTGGGTAAACCGTGAATATCG (SEQ ID NO: 12) to create an overlap for lacA R and the spectinomycin fragment resulting in a fragment of ca. 2200 bp.

For the construction of pBS2 these elements were subsequently combined in the following way:

Cloning of lacA F fragment into pUC18 using PstI and XbaI. Cloning of the spectinomycin lacA R fragment into the previously obtained plasmid using KpnI and EcoRI. Cloning of the linker using BamHI and KpnI into the previously obtained plasmid. Cloning of PHPAII promoter element into the previously obtained plasmid using BamHI and SpeI to obtain pBS2. All cloning steps were carried out by ligation of the respective digested fragments and vectors and transformation of E. coli DH5 alpha with subsequent screening for positive transformants on medium containing 100 micrograms mL⁻¹ ampicillin. E. coli DH5 alpha can be obtained from e.g. Toyobo Co. Ltd., Japan.

CoA transferase (ca. 1300 bp, Kyoto Encyclopedia of Genes and Genomes (KEGG) entry Cbei_{—}3833 and Cbei_{—}3834) and acetoacetate decarboxylase (ca. 750 bp, KEGG entry Cbei_{—}3835) genes were amplified using C. beijerinckii NCIMB 8052 genomic DNA as a template and TAATAACTAGTAAGGAGGACATATGAATAAATTAGTAAAATTAACAGATTTAAAGCGCATTTTCAAAGATG G (SEQ ID NO: 13) and GCCGCACGCGTTCATATATCCATAATCTTTAAGTTATCTGGAATAATTAAATCTGC (SEQ ID NO: 14) or TAATAACGCGTAAGGAGGACATATGTTAGAAAGTGAAGTATCTAAACAAATTACAACTCC (SEQ ID NO: 15) and GCCGCAGGCCTTTATTTTACTGAAAGATAATCATGTACAACCTTAGG (SEQ ID NO: 16) as primers, respectively. C. beijerinckii NCIMB 8052 can be obtained from e.g. the American Type Culture Collection (ATCC), USA. After MluI digestion, the fragments were ligated and the fragment of ca. 2000 bp corresponding to transferase and decarboxylase was obtained from this ligation mix by PCR using TAATAACTAGTAAGGAGGACATATGAATAAATTAGTAAAATTAACAGATTTAAAGCGCATTTTCAAAGATG G (SEQ ID NO: 13) and GCCGCAGGCCTTTATTTTACTGAAAGATAATCATGTACAACCTTAGG (SEQ ID NO: 16). Subsequently, this fragment was ligated with pBS2 using SpeI and StuI. Lastly using CAAACTGCAGGTGATGTCAAAGCTTGAAAAAACGCACG (SEQ ID NO: 1) and CGCGGAATTCCTAATGTGTGTTTACGACAATTCTCACTTCATACTTTTCC (SEQ ID NO: 4) a PCR fragment lacA [ctfAB adc] of ca. 5700 bp containing lacA F, PHPAII promoter, ctfAB, adc, spectinomycin fragment and lacA R was obtained from the ligation mixture. It was used to transform Bacillus subtilis 168 bp means of a standard transformation protocol and transformants were screened on medium containing 100 micrograms mL⁻¹spectinomycin.

The introduction of ipadh into the amyE locus of Bacillus subtilis 168 was carried out as described in the following. A region of ca. 1200 bp containing the ORF of ipadh (described in GenBank accession number AF157307) followed by an untranslated region (SEQ ID NO: 43) was amplified from C. beijerinckii NRRL B593 genomic DNA using oligonucleotides GGAGTCACTAGTAAGGAGGACATATGAAAGGTTTTGCAATGCTAGGTATTAATAAGTTAGG (SEQ ID NO: 17) and ATCTCGAGTTATAATATAACTACTGCTTTAATTAAGTCTTTTGGCTTG (SEQ ID NO: 18). C. beijerinckii NRRL B593 can be obtained from VTT Culture Collection, Finland. A region of ca. 1300 bp (SEQ ID NO: 19) conferring resistance to chloramphenicol was amplified from pHT01 using TATATTCTCGAGCGATCGGCAATAGTTACCCTTAT (SEQ ID NO: 20) and CAGTTATCTAGAGTCGATTTTTGTGATGCTCGT (SEQ ID NO: 21). pHT01 can be obtained from MoBiTec GmbH, Germany. A region of ca. 1000 bp termed amyE F was amplified from Bacillus subtilis 168 genomic DNA using ACTGGCATCAATTGCGTTATTCGCTGGATTTTTATTG (SEQ ID NO: 22) and ATGAAGGTACCGCGTACTGCCTGAACGAGAAGCTA (SEQ ID NO: 23). A region of ca. 1000 bp termed amyE R was amplified from Bacillus subtilis 168 genomic DNA using TAGAGTCTAGAGCGGAAATGGTGTGAGGTT (SEQ ID NO: 24) and AGTAAGCTTACCATTTAGCACGTAATCAAAGC (SEQ ID NO: 25). pUC18 was digested with EcoRI and KpnI and ligated with amyE F which had been digested with MfeI and KpnI. The obtained plasmid was digested with HindIII and XbaI and ligated with amyE R which had been digested with HindIII and XbaI. All cloning steps were carried out by ligation of the respective digested fragments and vectors and transformation of E. coli DH5 alpha with subsequent screening for positive transformants on medium containing 100 micrograms mL⁻¹ ampicillin. pBS2 was digested with SpeI and StuI and ligated with ipadh which had been digested with SpeI. From this ligation mix a DNA fragment of ca. 1500 bp containing PHPAII promoter and ipadh was obtained using TCCTGGTACCGATCTTCTCAAAAAATACTACCTGTCCCTTGC (SEQ ID NO: 26) and ATCTCGAGTTATAATATAACTACTGCTTTAATTAAGTCTTTTGGCTTG (SEQ ID NO: 18). This fragment was digested with XhoI and ligated with the aforementioned chloramphenicol resistance conferring DNA fragment which had been digested with XhoI. From this ligation mix a DNA fragment of ca. 2800 bp containing the PHPAII promoter, ipadh and the DNA fragment conferring chloramphenicol resistance was obtained using TCCTGGTACCGATCTTCTCAAAAAATACTACCTGTCCCTTGC (SEQ ID NO: 26) and CAGTTATCTAGAGTCGATTTTTGTGATGCTCGT (SEQ ID NO: 21). This fragment was digested with KpnI and XbaI and ligated with the aforementioned amyE F and R carrying pUC18 which had been digested with KpnI and XbaI. From this ligation mix a DNA fragment amyE [ipadh] of ca. 4800 bp carrying the PHPAII promoter, ipadh and the DNA fragment conferring chloramphenicol resistance flanked by amyE F and amyE R was obtained using ACTGGCATCAATTGCGTTATTCGCTGGATTTTTATTG (SEQ ID NO: 22) and AGTAAGCTTACCATTTAGCACGTAATCAAAGC (SEQ ID NO: 25). This fragment was used to transform Bacillus subtilis 168 and transformants were screened on medium containing 10 micrograms mL⁻¹ chloramphenicol.

thiL (ca. 1200 bp, KEGG entry CA_P0078) was amplified from C. acetobutylicum ATCC 824 genomic DNA using primers TAATAGCTAGCAAGGAGGACATATGAGAGATGTAGTAATAGTAAGTGCTGTAAGAACTGC (SEQ ID NO: 27) and GCCGCAGGCCTTAATAACTTAGTTATATATAACTATTTAGTCTCTTTCAACTACG (SEQ ID NO: 28). C. acetobutylicum ATCC 824 can be obtained from e.g. the American Type Culture Collection (ATCC), USA. After digestion with NheI and StuI the fragment was ligated with pBS2, which had been previously digested with SpeI and StuI. From this ligation, the PHPAII thiL terminator fragment of ca. 1500 bp was obtained using TCCTGGATCCGATCTTCTCAAAAAATACTACCTGTCCCTTGC (SEQ ID NO:6) and TCCGGGATCCAAAAAAGCCCGCTCATTAGG (SEQ ID NO: 29). This fragment was digested with BamHI and subsequently ligated with BamHI digested pHY300PLK to obtain pHY [thiL] and used to transform E. coli DH5 alpha followed by screening of positive transformants on medium containing 100 micrograms mL⁻¹ ampicillin. pHY300PLK can be obtained from Takara Bio Inc., Japan. Plasmid pHY [thiL] extracted from this E. coli strain was used to transform Bacillus subtilis 168 and transformants were screened on medium containing 10 micrograms mL⁻¹ tetracycline. Subsequent transformation of Bacillus subtilis 168 with above mentioned lacA [ctfAB adc], amyE [ipadh] and pHY [thiL] plasmid resulted in Bacillus subtilis [IPA].

pHY[Acetone n4] was constructed from a synthetic DNA fragment encoding for 4 polypeptides in for Bacillus subtilis 168 codon optimized form. Synthetic codon optimized DNA fragments can be obtained from e.g. DNA 2.0 Inc., USA. The polypeptides correspond to C. acetobutylicum ATCC 824 thiolase (SEQ ID NO: 30), C. beijerinckii NCIMB 8052 CoA transferase subunit A (SEQ ID NO: 31) and B (SEQ ID NO: 32) and acetoacetate decarboxylase (SEQ ID NO: 33). A ribosome binding site sequence and spacer AAGGAGGACAT is preceding each individual ORE. The 4 ORES are preceded by a DNA fragment containing a PHPAII promoter element from plasmid pUB110. The 4 polypeptides are followed by the terminator sequence GCAGCCCGCCTAATGAGCGGGCTTTTTT. The fragment of promoter element, 4 ORES and terminator sequence of ca. 3500 bp was amplified by PCR using primers TCCTGGATCCGATCTTCTCAAAAAATACTACCTGTCCCTTGC (SEQ ID NO: 6) and TCCGGGATCCAAAAAAGCCCGCTCATTAGG (SEQ ID NO: 29) introducing BamHI sites at either end of the sequence. Subsequently, the fragment was digested with BamHI and ligated with BamHI digested vector pHY300PLK to obtain pHY[Acetone n4] which was used to transform E. coli DH5 alpha. After plasmid extraction from a positive E. coli transformant screened on medium containing 100 micrograms mL⁻¹ ampicillin, pHY[Acetone n4] was used to transform Bacillus subtilis 168 to obtain Bacillus subtilis pHY[Acetone n4] after screening on medium containing 10 micrograms mL⁻¹ tetracycline.

pHY[Acetone n7] was constructed by individual PCR amplification of aforementioned 4 ORES using primers to introduce an extended spacing between ribosome binding site and initiation codon resulting in fragments that correspond in size to the aforementioned Clostridial genes. The sequence preceding the individual initiation codons was changed from AAGGAGGACAT to AAGGAGGAAAAAAA. The primers for the PCR reaction were designed in a way to be suitable for subsequent one step construction of pHY[Acetone n7] from pHY[Acetone n4] digested by SpeI and StuI containing aforementioned promoter element as well as the terminator sequence and the 4 ORFS with extended spacing by means of Clontech InFusion reaction. The primers used for this purpose were CATTTGTGCCAACTAGTTAAGGAGG TGAGGGACGTAGTAATAGTAAGCGCAGTCAG (SEQ ID NO: 34) and CTTAAAGCTTTTAGTCACGCTCAACGACCAATG (SEQ ID NO: 35) for the fragment corresponding to thiL, CGTGACTAAAAGCTTTAAGGAGG TGAACAAATTAGTCAAACTCACAGACCTAAAAAGAATATT CAAGG (SEQ ID NO: 36) and CTTAACGCGTTCATGCCGCCTCTTTC (SEQ ID NO: 37) corresponding to CoA transferase subunit A, GCGGCATGAACGCGTTAAGGAGG TGATCGTAGACAAAGTACTGGCCAAGGAG (SEQ ID NO: 38) and CTTAGCTAGCTCAGATATCCATAATCTTCAGGTTGTCC (SEQ ID NO: 39) corresponding to CoA transferase subunit B as well as GATATCTGAGCTAGCTAAGGAGG TGCTCGAATCAGAAGTCAGTAAACAAATCACAAC (SEQ ID NO: 40) and TGCATGCCTGCAGGTCGACTTACTTAACGCTCAGATAGTCATGGAC (SEQ ID NO: 41) corresponding to acetoacetate decarboxylase.

This InFusion reaction was used to transform E. coli DH5 alpha. After plasmid extraction from a positive E. coli transformant screened on medium containing 100 micrograms mL⁻¹ ampicillin, pHY[Acetone n7] was used to transform Bacillus subtilis 168 to obtain Bacillus subtilis pHY[Acetone n7] after screening on medium containing 10 micrograms mL⁻¹ tetracycline.

The [lacA Acetone n7] fragment was constructed from pHY[Acetone n7] and pBS2. pHY[Acetone n7] was digested with SpeI and StuI and the released ca. 3200 bp fragment containing the 4 ORFs corresponding to thiL, CoA transferase subunit A, CoA transferase subunit B and acetoacetate decarboxylase and carrying the extending ribosome binding site spacing was obtained. This fragment was then ligated with pBS2 digested with SpeI and StuI. Subsequently, the ca. 6500 bp [lacA Acetone n7] fragment was amplified using CAAACTGCAGGTGATGTCAAAGCTTGAAAAAACGCACG (SEQ ID NO: 1) and CGCGGAATTCCTAATGTGTGTTTACGACAATTCTCACTTCATACTTTTCC (SEQ ID NO: 4) and used to transform Bacillus subtilis168 and transformants were screened on medium containing 100 micrograms mL⁻¹ spectinomycin.

pHY[ipadh] was constructed by ligation of a synthetic DNA fragment into pHY300PLK using BamHI restriction sites. The synthetic DNA fragment consisted of a PHPAII promoter, ribosome binding site and spacer AAGGAGGAAAAAAA, an ORF encoding for C. beijerinckii NCIMB 8052 ipadh in for Bacillus subtilis 168 codon optimized form (SEQ ID NO: 42), an untranslated region (SEQ ID NO: 43) and a terminator sequence GCAGCCCGCCTAATGAGCGGGCTTTTTT. Transformation of E. coli DH5 alpha with this plasmid was followed by screening of clones on medium containing 100 micrograms mL⁻¹ ampicillin. The obtained plasmid from a positive clone was used to transform Bacillus megaterium DSM319 followed by screening on medium containing 10 micrograms mL⁻¹ tetracycline. Bacillus megaterium DSM319 can be obtained from the Deutsche Sammlung von Mikroorganismen and Zellkulturen GmbH, Germany.

### Examples 2

### Construction of Gene Disruptions in Bacillus subtilis

For the disruption of alsSD, a ca. 1200 bp DNA fragment (described in GenBank accession number M19465.1) encoding for a region conferring resistance to kanamycin, was amplified by PCR from pUB110 using the primers TAAATATAAATCGGATCCACAATCGGCAATTGACGAAAC (SEQ ID NO: 44) and ATACTATGTCGACCCAACATGATTAACAATTATTAGAGGTCATCGT (SEQ ID NO: 45). Two ca. 1000 bp fragments alsSD F and alsSD R were obtained by PCR using Bacillus subtilis genomic DNA as a template and primers GCTGAATTCAACCAATCACCATTTGATATTCTGT (SEQ ID NO: 46), TATGGATCCGTCCTTTATCTTGTAAAGCGTCAAA (SEQ ID NO: 47), ATACTATGTCGACGTTTTTGACTATGTGCTTGAGGATT (SEQ ID NO: 48) and TATGTTGCATGCTATTATCGCAGTGAAAACCAATACA (SEQ ID NO: 49), respectively. Subsequently, these fragments were cloned into pUC18 using restriction sites EcoRI, BamHI, SalI and SphI, respectively. All cloning steps were carried out by ligation of the respective digested fragments and vectors and transformation of E. coli DH5 alpha with subsequent screening for positive transformants on medium containing 100 micrograms mL⁻¹ ampicillin. From this plasmid a linear DNA fragment of ca. 3200 bp was obtained by PCR using primers GCTGAATTCAACCAATCACCATTTGATATTCTGT (SEQ ID NO: 46) and TATGTTGCATGCTATTATCGCAGTGAAAACCAATACA (SEQ ID NO: 49) and was used to transform Bacillus subtilis 168 according to a well established method. Transformants were screened on medium containing 30 micrograms mL⁻¹ kanamycin and Bacillus subtilis delta-alsSD was confirmed through PCR of the alsSD locus.

For the construction of pMUTIN4[pta], a ca. 600 bp fragment corresponding to an internal part of the pta ORF was amplified from the genomic DNA of Bacillus subtilis 168 using the primers TAGAGAAGCTTGCAGGAAACAAAGTGCTGAATCCGATTG (SEQ ID NO: 50) and TAGAGGGATCCGCAGCATCAAATTGGAATTCGCCGTC (SEQ ID NO: 51). Subsequently, pMUTIN4 and the ca. 600 bp fragment were digested with HindIII and BamHI, ligated and used to transform E. coli DH5 alpha. pMUTIN4 can be obtained from the Bacillus Genetic Stock Center, USA. After plasmid extraction from a positive E. coli transformant screened on medium containing 100 micrograms mL⁻¹ ampicillin, pta was disrupted in Bacillus subtilis 168 bp transforming it with pMUTIN4[pta] and screening for transformants on medium containing 0.5 micrograms mL⁻¹ erythromycin. The integration of pMUTIN4[pta] into the Bacillus subtilis 168 genome was confirmed by PCR.

For the construction of pMUTIN4[ccpA] a ca. 700 bp fragment corresponding to an internal part of the ccpA ORF was amplified from the genomic DNA of Bacillus subtilis 168 using the primers GTACAAGCTTATGAGCAATATTACGATCTACGATGTAGCGAGAGAAG (SEQ ID NO: 52) and GTTTGGATCCGCAGTGCTTCGAGTCCGGAATCATATGTGTAATC (SEQ ID NO: 53). Subsequently, pMUTIN4 and the ca. 700 bp fragment were digested with HindIII and BamHI, ligated and used to transform E. coli DH5 alpha. After plasmid extraction from a positive E. coli transformant, screened on medium containing 100 micrograms mL⁻¹ ampicillin, ccpA was disrupted in Bacillus subtilis 168 bp transforming it with pMUTIN4[ccpA] and screening for transformants on medium containing 0.5 micrograms mL⁻¹ erythromycin. The integration of pMUTIN4 into the Bacillus subtilis 168 genome was confirmed by PCR.

### Examples 3

### Production of Acetone by Bacillus subtilis in Shake Flask

Pre-cultures of B. subtilis [lacA Acetone n7], B. subtilis [lacA Acetone n7] delta-alsSD, B. subtilis pHY[Acetone n4], B. subtilis pHY[Acetone n4] delta-alsSD, B. subtilis pHY[Acetone n7] and B. subtilis pHY[Acetone n7] delta-alsSD were cultured for 24 h at 30 degrees Celsius and 280 rpm in a 14 mL round bottom tube in 5 mL of CSE medium plus supplements as listed in Table 2. For tube or flask culture, no Adekanol was used. 10 micrograms mL⁻¹ tetracycline was added to the cultures of B. subtilis pHY[Acetone n4], B. subtilis pHY[Acetone n4] delta-alsSD, B. subtilis pHY[Acetone n7] and B. subtilis pHY[Acetone n7] delta-alsSD. These strains carried genes encoding for thiolase, CoA transferase and acetoacetate decarboxylase in for Bacillus subtilis codon optimized form according to Example 1. Subsequently, 200 microliters of this culture was used to inoculate 20 mL of the same medium in a 125 mL baffled shake flask. The main culture was carried out at 30 degrees Celsius and 200 rpm. The experiment was carried out in triplicates. The data presented here will be of the form value plus or minus 95% confidence interval.

After 40 h the following yields of acetone on glucose were determined:

The yields were calculated by dividing the concentration in [g/L] of acetone in the growth medium after 40 h by the concentration of consumed glucose in [g/L] after 40 h to obtain a yield in [g/g]. B. subtilis [lacA Acetone n7] 0.0025 plus or minus 0.0005 g g⁻¹, B. subtilis [lacA Acetone n7] delta-alsSD 0.0116 plus or minus 0.0012 g B. subtilis pHY[Acetone n4] 0.0067 plus or minus 0.0006 g g⁻¹, B. subtilis pHY[Acetone n4] delta-alsSD 0.0287 plus or minus 0.0018 g g⁻¹, B. subtilis pHY[Acetone n7] 0.0026 plus or minus 0.0005 g g⁻¹ and B. subtilis pHY[Acetone n7] delta-alsSD 0.0075 plus or minus 0.0008 g g⁻¹. B. subtilis [lacA Acetone n7] delta-alsSD shows statistically significant higher yield than B. subtilis pHY[Acetone n7] delta-alsSD (p=0.0249). B. subtilis pHY[Acetone n4] delta-alsSD shows statistically significant higher yield than B. subtilis [lacA Acetone n7] delta-alsSD (p=0.0009).

Furthermore, the combined yield of lactate, acetate, acetoin and 2,3-butanediol on glucose were as follows: B. subtilis [lacA Acetone n7] 0.402 plus or minus 0.059 g g⁻¹, B. subtilis [lacA Acetone n7] delta-alsSD 0.612 plus or minus 0.013 g g⁻¹, B. subtilis pHY[Acetone n4] 0.481 plus or minus 0.031 g g⁻¹, B. subtilis pHY[Acetone n4] delta-alsSD 0.494 plus or minus 0.034 g g⁻¹, B. subtilis pHY[Acetone n7] 0.537 plus or minus 0.042 g g⁻¹ and B. subtilis pHY[Acetone n7] delta-alsSD 0.583 plus or minus 0.015 g g⁻¹.

TABLE 1
Biomass, Metabolites and Yields of Different Acetone Producing B. subtilis

| | | lacA | | pHY | | pHY |
|---|---|---|---|---|---|---|
| | lacA | [Acetone n7] | pHY | [Acetone n4] | pHY | [Acetone n7] |
| | [Acetone n7] | ΔalsSD | [Acetone n4] | ΔalsSD | [Acetone n7] | ΔalsSD |
| Glucose Consumed [g/L] | 23.000 ± 0.000 | 11.647 ± 0.403 | 23.000 ± 0.000 | 12.009 ± 1.592 | 22.123 ± 1.718 | 12.514 ± 0.713 |
| OD 660 nm [AU] | 18.162 ± 0.583 | 8.705 ± 0.471 | 14.907 ± 2.779 | 9.533 ± 1.734 | 14.648 ± 2.029 | 8.650 ± 0.439 |
| OD/Glucose [AU L/g] | 0.790 ± 0.025 | 0.749 ± 0.066 | 0.648 ± 0.121 | 0.801 ± 0.166 | 0.663 ± 0.081 | 0.693 ± 0.068 |
| Lactate [g/L] | 0.000 ± 0.000 | 0.512 ± 0.240 | 0.000 ± 0.000 | 0.068 ± 0.132 | 0.545 ± 0.549 | 0.000 ± 0.000 |
| Acetate [g/L] | 1.526 ± 0.196 | 6.615 ± 0.112 | 2.023 ± 0.058 | 5.843 ± 0.549 | 2.464 ± 0.068 | 7.296 ± 0.239 |
| Acetoin/2,3-Butanediol [g/L] | 7.724 ± 1.170 | 0.000 ± 0.000 | 9.049 ± 0.677 | 0.000 ± 0.000 | 8.844 ± 0.637 | 0.000 ± 0.000 |
| Byproducts*/Glucose [g/g] | 0.402 ± 0.059 | 0.612 ± 0.013 | 0.481 ± 0.031 | 0.494 ± 0.034 | 0.537 ± 0.042 | 0.583 ± 0.015 |
| Acetone [g/L] | 0.058 ± 0.012 | 0.134 ± 0.009 | 0.154 ± 0.013 | 0.343 ± 0.027 | 0.058 ± 0.015 | 0.094 ± 0.016 |
| Acetone/Glucose [g/g] | 0.0025 ± 0.0005 | 0.0116 ± 0.0012 | 0.0067 ± 0.0006 | 0.0287 ± 0.0018 | 0.0026 ± 0.0005 | 0.0075 ± 0.0008 |

*Byproducts refer to the sum of lactate, acetate, acetoin and 2,3-butanediol.

From the data summarized in Table 1, it can be furthermore seen that disruption of alsSD has a number of effects. It firstly causes the Bacillus cell to mainly produce acetate as a byproduct and no acetoin or 2,3-butanediol. Furthermore, it unexpectedly changes the Bacillus cell to increase the yield of the sum byproducts (lactate, acetate, acetoin and 2,3-butanediol) on glucose. Disruption of alsSD, therefore, increases titer and yield of acetone on glucose not by lowering the sum of byproduct yield, but by unexpected unknown effect. This effect of alsSD disruption is significant regardless of the variation in genetic construction of the acetone producing Bacillus subtilis.

### Example 4

### Production of Isopropyl Alcohol Using Bacillus subtilis in 1 L Culture Vessel

Bacillus subtilis and Bacillus subtilis delta-alsSD were transformed consecutively with lacA [ctfAB adc], amyE [ipadh] and pHY [thiL] to obtain B. subtilis [IPA] and B. subtilis delta-alsSD [IPA]. Additionally, B. subtilis delta-alsSD [IPA] was transformed with pMUTIN4[pta] to obtain B. subtilis delta-alsSD delta-pta [IPA]. These strains carried genes encoding for thiolase, CoA transferase, acetoacetate decarboxylase and isopropyl alcohol dehydrogenase in form of Clostridial DNA nucleotide sequence without further codon modification according to Example 1. These three strains were used for acetone and isopropyl alcohol production in a 1 L jar fermenter manufactured by Able Corporation, Japan according to the following method. A pre-culture of each strain was cultured for 24 h at 30 degrees Celsius and 280 rpm in a 14 mL round bottom tube in 5 mL of CSE medium plus supplements as listed in Table 2. In all cases 10 micrograms mL⁻¹ tetracycline was added to the culture. In the case of B. subtilis delta-alsSD delta-pta [IPA] 0.5 micrograms mL⁻¹ erythromycin was additionally added. Subsequently, the entire pre-culture was used to inoculate 495 mL of the same medium in a 1 L jar fermenter manufactured by Able Corporation, Japan. The conditions of the jar fermentation were 30 degrees Celsius, 550 rpm, 0.3 v/vm (air). The pH was automatically controlled to be pH 7 through the addition of a 14% (v/v) solution of ammonia in water. The unit % (v/v) referring to volume concentration in percent. After 2 h of culture, a feed of 50% (w/w) glucose was started at a rate of 0.02 g min⁻¹. The unit % (w/w) referring to mass fraction in percent. The off gas line was connected to 2 water traps of 800 mL each to trap acetone and isopropyl alcohol that was formed during the process.

TABLE 2
Medium Composition for Isopropyl Alcohol Production in B. subtilis

| Component | Amount | |
|---|---|---|
| K₂HPO₄ | 70 | mM |
| KH₂PO₄ | 30 | mM |
| (NH₄)₂SO₄ | 25 | mM |
| MgSO₄ | 0.5 | mM |
| MnSO₄ | 0.01 | mM |
| Fe(III) ammonium citrate | 22 | mg L⁻¹ |
| Na-Succinate | 6 | g L⁻¹ |
| K-Glutamate | 8 | g L⁻¹ |
| Casein Hydrolysate | 5 | g L⁻¹ |
| Glucose | 23 | g L⁻¹ |
| L-Tryptophan | 50 | mg L⁻¹ |
| Zn²⁺ | 1.3 | μM |
| Trace element solution | 1 X | |
| Antibiotics | As needed | |
| Adekanol | 1 | mL L⁻¹ |

Samples were analyzed using established methods of HPLC and GC-FID. HPLC analysis was carried out on a WATERS e2695 Separations Module equipped with 2489 UV/visible Detector, 2414 Refractive Index Detector, column oven and a PC running Empower 2 software. An ULTRON PS-80H column was installed. The oven temperature was set to 60 degrees Celsius and the flow rate was 1 mL min⁻¹. Elution was carried out isocratically using nanopure H₂O that had been adjusted to pH 2.1 with HClO₄.

GC analysis was carried out on an Agilent Technologies 7890A gas chromatograph with FID detector and split/splitless inlet. A DB-624 column (JW-Technologies) with 30 m length, 0.25 mm ID and 1.4 micro m film thickness was used. The inlet temperature was set to 250 degrees Celsius and the FID temperature to 300 degrees Celsius. Helium was used as a carrier gas at a pressure of 98.2 kPa. Samples were injected as 0.6 micro L at a split ratio of 30:1. The initial oven temperature of 40 degrees Celsius was held for 5 minutes and subsequently ramped at 10 degrees Celsius per minute to 250 degrees Celsius with a total run time of 26 min.

Formation of isopropyl alcohol, acetone, 2,3-butanediol, acetoin and acetate are summarized in Table 3.

TABLE 3
Metabolites of Isopropyl Alcohol Production by Different B. subtilis

| | B. subtilis | B. subtilis | B. subtilis |
|---|---|---|---|
| Compound/[g L⁻¹] | [IPA] | ΔalsSD [IPA] | ΔalsSD Δpta [IPA] |
| Isopropyl alcohol | 0.008 | 1.8 | 2.4 |
| Acetone | 0.1 | 2.2 | 2.3 |
| 2,3-Butanediol | 17 | 0 | 0 |
| Acetoin | 40 | 0 | 0 |
| Acetate | 1 | 29 | 31 |

Similar to Example 3, disruption of alsSD resulted in shift in byproduct from acetoin to acetate accompanied by an unexpected increase in isopropyl alcohol and acetone titer.

Presecan-Siedel et al. describe a reduction of acetate formation in B. subtilis delta-alsSD from 27 to 9 mM upon disruption of pta (Presecan-Siedel et al. Journal of Bacteriology 1999 181(22) 6889-6897). In contrast to this description disruption of pta unexpectedly did not result in a reduction in acetate formation but rather in an increase in isopropyl alcohol production in the isopropyl alcohol producing Bacillus of this example.

### Example 5

### Production of Acetone Using Bacillus subtilis in 1 L Culture Vessel

Bacillus subtilis delta-alsSD was transformed with pHY[Acetone n4], pHY[Acetone n7] and lacA[Acetone n7] using a well established transformation method. These strains carried genes encoding for thiolase, CoA transferase and acetoacetate decarboxylase in for Bacillus subtilis codon optimized form according to Example 1. The three strains were used for acetone production in a 1 L jar fermenter manufactured by Able Corporation, Japan according to the conditions described in Example 4. 10 micrograms mL⁻¹ tetracycline was added to the cultures of B. subtilis pHY[Acetone n4] delta-alsSD and B. subtilis pHY[Acetone n7] delta-alsSD.

The highest acetone titer was 4.6 g L⁻¹ for pHY[Acetone n4] (at 140 h), 2.8 g L⁻¹ for pHY[Acetone n7] (at 120 h) and 8.9 g L⁻¹ for lacA[Acetone n7] (at 140 h). Acetone titer for pHY[Acetone n7] was 2.4 g L¹ (at 140 h). Reduction of acetone titer in the case of pHY[Acetone n7] is presumably due to the rate of acetone evaporation exceeding the rate of acetone formation after 120 h. In this example, most preferable strain for acetone production is lacA[Acetone n7] followed by pHY[Acetone n4] followed by pHY[Acetone n7].

### Example 6

### Product Profile from Glucose of Bacillus subtilis Delta-ccpA Delta-alsSD

A pre-culture of Bacillus subtilis delta-alsSD delta-ccpA and Bacillus subtilis delta-alsSD was cultured for 24 h at 30 degrees Celsius and 280 rpm in a 14 mL round bottom tube in 5 mL of CSE medium plus supplements as listed in Table 2. 0.5 micrograms mL⁻¹ erythromycin was additionally added. For tube or flask culture, no Adekanol was used. Subsequently, 200 microliters of this culture was used to inoculate 20 mL of the same medium in a 125 mL baffled shake flask. The main culture was carried out at 30 degrees Celsius and 200 rpm. After 40 h of culture Bacillus subtilis delta-alsSD delta-ccpA had consumed 2% (w/w) glucose and produced 0 g L⁻¹ of 2,3-butanediol, 0 g L⁻¹ of acetoin, 0 g L⁻¹ of acetate and 0 g L of lactate. In comparison, Bacillus subtilis delta-alsSD had consumed 1.03% (w/w) glucose and produced 0 g L⁻¹ of 2,3-butanediol, 0 g L⁻¹ of acetoin, 7.5 g L⁻¹ of acetate and 0 g L⁻¹ of lactate. The unit % (w/w) referring to mass fraction in percent. Turinsky et al. describe a reduction of acetate in Bacillus subtilis from 13 to 7.3 mM upon disruption of ccpA when grown on glucose (Turinsky et al. Journal of Bacteriology 2000 182(19) 5611-5614. In contrast to this result and unexpected from the effect of alsSD disruption described in Examples 3 and 4, the combined disruption of alsSD and ccpA caused a complete reduction of acetate to 0 g L under the given conditions.

### Example 7

### Production of Acetone/Isopropyl Alcohol Using Bacillus subtilis Delta-ccpA Delta-alsSD

This example is comparing the production of acetone and isopropyl alcohol as well as the formation of by-products between B. subtilis delta-alsSD [IPA] and B. subtilis delta-ccpA delta-alsSD lacA[Acetone n7] under the same conditions as outlined in Example 4. 0.5 micrograms mL⁻¹ erythromycin was additionally added in the case of B. subtilis delta-ccpA delta-alsSD lacA[Acetone n7]. This condition is referred to as “standard nitrogen source” condition. Additionally, the production of acetone and isopropyl alcohol as well as the formation of by-products between B. subtilis delta-alsSD [IPA] and B. subtilis delta-alsSD delta-ccpA [IPA*] is compared under the same conditions as outlined in Example 4 with the exception that instead of a feed of 50% (w/w) glucose a feed of 50% (w/w) glucose 5% (w/w) casein hydrolysate was applied. The unit % (w/w) referring to mass fraction in percent. This condition is referred to as “increased nitrogen source” condition. 0.5 micrograms mL⁻¹ erythromycin was additionally added in the case of B. subtilis delta-ccpA delta-alsSD [IPA*]. The results are summarized in Table 4. B. subtilis delta-alsSD [IPA] carried genes encoding for thiolase, CoA transferase, acetoacetate decarboxylase and isopropyl alcohol dehydrogenase in form of Clostridial DNA nucleotide sequence without codon modification. B. subtilis delta-ccpA lacA[Acetone n7] carried genes encoding for thiolase, CoA transferase and acetoacetate decarboxylase in for B. subtilis codon optimized form. B. subtilis delta-alsSD delta-ccpA [IPA*] carried genes encoding for thiolase, CoA transferase and acetoacetate decarboxylase in for Bacillus subtilis codon optimized form and a gene encoding for isopropyl alcohol dehydrogenase in form of Clostridial DNA nucleotide sequence without codon optimization according to Example 1.

TABLE 4
C3 and Acetate Formation by Different B. subtilis
under Two Nitrogen Source Conditions

| | | B. subtilis | | B. subtilis |
|---|---|---|---|---|
| | B. subtilis | ΔalsSD | B. subtilis | ΔalsSD |
| | ΔalsSD | ΔccpA lacA | ΔalsSD | ΔccpA |
| | [IPA] | [Acetone n7] | [IPA] | [IPA*] |
| Compound/[g L⁻¹] | Standard nitrogen source | | Increased nitrogen source | |
| C3 | 4 | 0.25 | 5.2 | 4.4 |
| (Isopropyl alcohol + | | | | |
| acetone) | | | | |
| Acetate | 29 | 0 | 25 | 13 |

The data in Table 4 shows that acetone or isopropyl alcohol producing B. subtilis delta-alsSD delta-ccpA can reach comparable levels of combined isopropyl alcohol and acetone to B. subtilis delta-alsSD [IPA] if availability of nitrogen source is increased. In contrast, increased nitrogen source availability has only a small effect on B. subtilis delta-alsSD [IPA]. Acetate concentration using B. subtilis delta-alsSD delta-ccpA [IPA*] under increased nitrogen source condition was reduced to about half compared to B. subtilis delta-alsSD [IPA].

### Example 8

### Production of Acetone Using Bacillus megaterium DSM 319

For the production of acetone using Bacillus megaterium DSM 319 a pre culture of 5 mL C medium plus nitrogen source and supplements (Table 5) is inoculated and grown over night at 30 degrees Celsius and 280 rpm on a rotary shaker in a 14 mL round bottom culture tube. For the production of acetone in shake flasks, 200 microliters of the pre culture is used to inoculate 20 mL of the main culture in a 125 mL baffled flask. The main culture is carried out at 30 degrees Celsius and 200 rpm on a rotary shaker.

TABLE 5
Medium Composition for Acetone Production in B. megaterium

| Component | Amount | |
|---|---|---|
| K₂HPO₄ | 70 | mM |
| KH₂PO₄ | 30 | mM |
| (NH₄)₂SO₄ | 25 | mM |
| MgSO₄ | 0.5 | mM |
| MnSO₄ | 0.01 | mM |
| Fe(III) ammonium citrate | 22 | mg L⁻¹ |
| Yeast extract | 5 | g L⁻¹ |
| Glucose | 20 | g L⁻¹ |

| | |
|---|---|
| Trace Element Solution | 1 X |

After 24 h the acetone titer in the medium was 80 mg L as determined by GC-FID. GC analysis was carried out on an Agilent Technologies 7890A gas chromatograph with FID detector and split/splitless inlet. A DB-624 column (JW-Technologies) with 30 m length, 0.25 mm ID and 1.4 micro m film thickness was used. The inlet temperature was set to 250 degrees Celsius and the FID temperature to 300 degrees Celsius. Helium was used as a carrier gas at a pressure of 98.2 kPa. Samples were injected as 0.6 micro L at a split ratio of 30:1. The initial oven temperature of 40 degrees Celsius was held for 5 minutes and subsequently ramped at 10 degrees Celsius per minute to 250 degrees Celsius with a total run time of 26 min.

### Example 9

### Construction of Recombinant Acetone Producing Bacillus megaterium

Protoplasts of Bacillus megaterium DSM319 were transformed with pHY[Acetone n7] according to an established method. After regeneration of protoplasts on appropriate medium containing 10 micrograms mL⁻¹ tetracycline, positive transformants were identified by plasmid extraction and analysis.

### Example 10

### Production of Acetone Using Recombinant Acetone Producing Bacillus megaterium

For the production of acetone using recombinant Bacillus megaterium of Example 9, a pre culture of 5 mL C medium plus nitrogen source and supplements (Table 5) is inoculated and grown over night at 30 degrees Celsius and 280 rpm on a rotary shaker in a 14 mL round bottom culture tube. 10 micrograms mL⁻¹ tetracycline was additionally added. For the production of acetone in shake flasks, 200 microliters of the pre culture is used to inoculate 20 mL of the main culture in a 125 mL baffled flask. In pre culture and main culture, tetracycline was added to a final concentration of 10 micrograms mL⁻¹. The main culture is carried out at 30 degrees Celsius and 200 rpm on a rotary shaker. After 24 h the acetone titer in the medium was 276 mg L⁻¹ as determined by GC-FID.

GC analysis was carried out on an Agilent Technologies 7890A gas chromatograph with FID detector and split/splitless inlet. A DB-624 column (JW-Technologies) with 30 m length, 0.25 mm ID and 1.4 micro m film thickness was used. The inlet temperature was set to 250 degrees Celsius and the FID temperature to 300 degrees Celsius. Helium was used as a carrier gas at a pressure of 98.2 kPa. Samples were injected as 0.6 micro L at a split ratio of 30:1. The initial oven temperature of 40 degrees Celsius was held for 5 minutes and subsequently ramped at 10 degrees Celsius per minute to 250 degrees Celsius with a total run time of 26 min.

This value is significantly higher than production by Bacillus megaterium DSM319 as described in Example 8.

### Example 11

### Construction of Recombinant Isopropyl Alcohol Producing Bacillus megaterium

Protoplasts of Bacillus megaterium DSM319 were transformed with pHY[ipadh] according to an established method. After regeneration of protoplasts on appropriate medium containing 10 micrograms mL⁻¹ tetracycline, positive transformants were identified by plasmid extraction and analysis.

### Example 12

### Production of Isopropyl Alcohol Using Recombinant Isopropyl Alcohol Producing Bacillus megaterium

For the production of isopropyl alcohol using recombinant Bacillus megaterium of Example 11 a pre culture of 5 mL C medium plus nitrogen source and supplements (Table 5) is inoculated and grown over night at 30 degrees Celsius and 280 rpm on a rotary shaker in a 14 mL round bottom culture tube. 10 micrograms mL⁻¹ tetracycline was additionally added. For the production of isopropyl alcohol in shake flasks, 200 microliters of the pre culture is used to inoculate 20 mL of the main culture in a 125 mL baffled flask. In pre culture and main culture, tetracycline was added to a final concentration of 10 micrograms mL⁻¹. The main culture is carried out at 30 degrees Celsius and 200 rpm on a rotary shaker.

After 24 h the acetone titer in the medium was 39 mg L and the isopropyl alcohol titer was 53 mg L⁻¹ as determined by GC-FID. GC analysis was carried out on an Agilent Technologies 7890A gas chromatograph with FID detector and split/splitless inlet. A DB-624 column (JW-Technologies) with 30 m length, 0.25 mm ID and 1.4 micro m film thickness was used. The inlet temperature was set to 250 degrees Celsius and the FID temperature to 300 degrees Celsius. Helium was used as a carrier gas at a pressure of 98.2 kPa. Samples were injected as 0.6 micro L at a split ratio of 30:1. The initial oven temperature of 40 degrees Celsius was held for 5 minutes and subsequently ramped at 10 degrees Celsius per minute to 250 degrees Celsius with a total run time of 26 min.

### [Sequence Listing]

MC-90170WO.ST25.txt

1. A microorganism of the genus Bacillus, comprising one or more gene(s) selected from a group consisting of a gene conferring thiolase activity, a gene conferring CoA transferase activity and a gene conferring acetoacetate decarboxylase activity,
wherein the microorganism produces acetone.

2. The microorganism according to claim 1,
wherein at least one of said genes is introduced into the microorganism in the following way:
A) integration of related gene(s) into a chromosome of the microorganism, or
B) provision of related gene(s) on a plasmid using a ribosome binding site to initiation codon spacing of four nucleotides.

3. The microorganism according to claim 1,
wherein all of said genes are introduced into the microorganism in the following way:
A) integration of related genes into a chromosome of the microorganism, or
B) provision of related genes on a plasmid using a ribosome binding site to initiation codon spacing of four nucleotides.

4. The microorganism according to claim 1,
wherein at least one of said genes are derived from the genus Clostridium.

5. The microorganism according to claim 1,
wherein an acetolactate synthase (alsS) activity or an alpha acetolactate decarboxylase (alsD) activity has been inactivated.

6. The microorganism according to claim 5,
wherein both of the acetolactate synthase (alsS) activity and the alpha acetolactate decarboxylase (alsD) activity have been inactivated.

7. The microorganism according to claim 1,
wherein a carbon catabolite repression protein A (ccpA) has been inactivated.

8. The microorganism according to claim 1,
wherein a phosphotransacetylase (pta aka eutD) activity has been inactivated.

9. The microorganism according to claim 1,
wherein the microorganism is derived from Bacillus subtilis.

10. The microorganism according to claim 1,
wherein the microorganism is derived from Bacillus megaterium.

11. The microorganism according to claim 1, further comprising a gene conferring isopropyl alcohol dehydrogenase activity,
wherein the microorganism produces isopropanol from acetone.

12. The microorganism according to claim 11,
wherein the genes conferring isopropyl alcohol dehydrogenase activity are derived from the genus Clostridium.

13. A method for producing acetone, comprising culturing the microorganism according to claim 1 to produce acetone.

14. The method for producing acetone according to claim 13,
wherein the step of culturing the microorganism includes culturing the microorganism in a medium while supplying a gas to a culturing mixture and collecting the acetone produced by the culturing mixture.

15. A method for producing isopropyl alcohol, comprising culturing the microorganism according to claim 11 to produce isopropyl alcohol.

16. The method for producing isopropyl alcohol according to claim 15,
wherein the step of culturing the microorganism includes culturing the microorganism in a medium while supplying a gas to a culturing mixture and collecting the isopropyl alcohol produced by the culturing mixture.

17. A method for producing acetone, comprising culturing Bacillus megaterium DSM319 to produce acetone.

18. A method for producing isopropyl alcohol, comprising culturing the microorganism according to claim 12 to produce isopropyl alcohol.

19. The method for producing isopropyl alcohol according to claim 18,
wherein the step of culturing the microorganism includes culturing the microorganism in a medium while supplying a gas to a culturing mixture and collecting the isopropyl alcohol produced by the culturing mixture.
